(19) United States
(12) Patent Application Publication
(10) Pub. No.: US 2010/0298628 A1
(43) Pub. Date: Nov. 25, 2010

(54) STRESS URINARY INCONTINENCE TREATMENT

(75) Inventors: William J. Taylor, Woodbury, MN (US); Kipling Thacker, Excelsior, MN (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: Lifecore Biomedical, LLC, Chaska, MN (US)

(21) Appl. No.: 12/647,900

(22) Filed: Dec. 28, 2009

Related U.S. Application Data
(60) Provisional application No. 61/142,032, filed on Dec. 31, 2008.

(51) Int. Cl. A61F 2/02; A61K 31/728; A61K 38/44; A61P 13/00

(52) U.S. Cl. 600/30; 514/54; 424/94.4

(57) ABSTRACT

A method for stabilizing a urethra in a female patient having stress urinary incontinence caused by stretching of a pubourethral ligament includes filling a substantial portion of the pubourethral space. The method includes injecting precursor materials for a cross-linked hyaluronic acid (HA) into a pubourethral space the PUL and allowing the precursor materials to form a cross-linked hyaluronic acid such that the cross-linked hyaluronic acid fills a substantial portion of the pubourethral space.

### CROSS-REFERENCE TO RELATED APPLICATION

The present application claims priority to U.S. Provisional Application Ser. No. 61/142,032, filed Dec. 31, 2008, entitled “STRESS URINARY INCONTINENCE TREATMENT” which is incorporated herein by reference in its entirety.

### TECHNICAL FIELD

The present invention relates to treatment of urinary incontinence, and in particular, to compositions and methods for treatment of stress urinary incontinence.

### BACKGROUND

The lower portion of the human urinary tract comprises a bladder and a urethra, with the urethra providing a fluid pathway for urine to exit the body from the bladder. Urinary continence is the ability to voluntarily control the flow of urine from the bladder through the urethra, and urinary continence is facilitated by a number of mechanisms in the lower portion of the urinary tract. For example, muscles such as sphincter muscles can be used to constrict flow out of the bladder. In addition, the urethra itself has a passive resistance to the flow of fluid. In the female anatomy, continence is also facilitated by body structures that support the urethra. For example, fascia and pubourethral ligaments (PULs) in a female support the urethra by anchoring the urethra to the structures of the pelvic wall, such as the puborectalis muscle or arcus tendineus fasciae pelvis.

Urinary incontinence is one of the most prevalent conditions of the lower urinary tract. A common type of urinary incontinence is stress urinary incontinence (SUI), a condition in which an acute stress (e.g., coughing, jumping, or some other sudden stress or movement) causes a temporary loss of continence. In some women, SUI results from a deficient fascia and/or PULs. The PULs, for example, may stretch to an increased length, such that they no longer adequately support the urethra. This increased length of the PULs may result in urethral hypermobility, which may contribute to intrinsic sphincter deficiency. The most prevalent treatment of a deficient PUL is the use of urethral slings. Treatment using urethral slings, however, requires a significant surgical procedure and is not effective for all women. Alternate devices, compositions and/or procedures are needed to address the issue of urinary incontinence.

### SUMMARY

The present invention, according to some embodiments is a method for stabilizing a urethra in a female patient having stress urinary incontinence. The method comprises injecting precursor materials for a cross-linked hyaluronic acid (HA) into a pubourethral space and allowing the precursor materials to form a cross-linked hyaluronic acid such that the cross-linked hyaluronic acid fills substantially the entire pubourethral space.

According to other embodiments, the invention includes a method for stabilizing a urethra in a female patient having stress urinary incontinence wherein a pubourethral ligament (PUL) has been stretched, creating slack in the PUL. The method comprises injecting an amount of curable material into a pubourethral space such that the curable material restricts mobility of the urethra. The material is cured, and the curable material has a first consistency when injected and a second, thicker consistency after the curable material is allowed to cure.

According to other embodiments, the invention includes a method for stabilizing a urethra in a female patient having stress urinary incontinence wherein a pubourethral ligament (PUL) has been stretched, creating slack in the PUL. The method comprises injecting a first hyaluronic acid (HA) into a pubourethral space, the first HA having a first level of cross-linking. The continence of the female patient is monitored and, if continence is improved with the introduction of the first HA, a second HA is injected into the pubourethral space after the first HA has at least partially degraded. The second HA has a second, greater level of cross-linking.

According to yet other embodiments, the invention includes a method for stabilizing a urethra in a female patient having stress urinary incontinence. The method comprises disposing a space filling material in a pubourethral space in contact with the urethra and/or PUL such that the space filling material restricts mobility of the urethra.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a female bladder and urethra;

FIG. 2 is a schematic cross-sectional view of a female urethra and the ligaments supporting the urethra;

FIG. 3 is a schematic cross-sectional view of a female urethra and the ligaments supporting the urethra where the ligaments have been stretched;

FIG. 4 is a schematic cross-sectional view of a female urethra and the ligaments supporting the urethra where a space filling material surrounds the urethra; and

FIG. 5 is a flow chart for a method of treating stress urinary incontinence.

### DETAILED DESCRIPTION

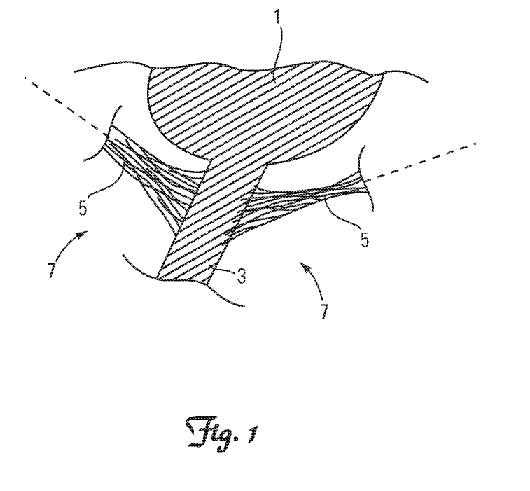

FIG. 1 shows a schematic view of a portion of the female urinary tract, including a bladder 1 and urethra 3. Also shown are the pubourethral ligaments (PULs) 5. The PULs 5 extend from the urethra 3, through the pubourethral space 7, to the pubic bone (i.e., the arcus tendineus fasciae). When the PULs 5 are relatively taught between the urethra 3 and the pubic bone, the PULs 5 restrict mobility of the urethra 3, facilitating continence.

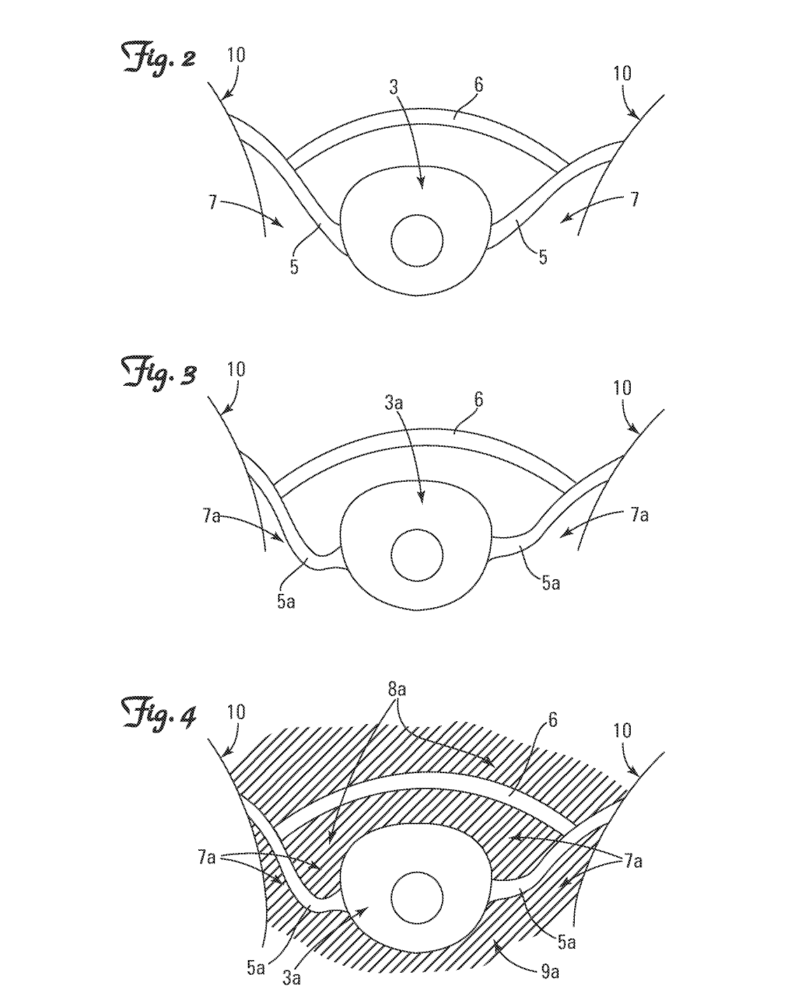

FIG. 2 shows a schematic cross-sectional view of the female urinary tract. Shown in this figure are healthy PULs 5 that extend in a relatively taught fashion from the urethra 3, through the pubourethral space 7, to the pubic bone 10. FIG. 2 also shows the periurethral ligament 6, which extends generally from one PUL 5 to another PUL 5 between the urethra 3 and the pelvis. In contrast, FIG. 3 shows a cross-sectional view of a portion of another female urinary tract in which the PULs 5a are deficient. As shown in this example, these deficient PULs 5a are relatively loose or stretched out as compared to a normal or healthy PUL 5. The stretching of the PULs can be caused by a number of factors including child birth or other stresses.

In some instances, deficient PULs 5a as shown in FIG. 3 allow the urethra 3a to be relatively mobile (often called “hypermobility”) within the pubourethral space 7a, for example when the female is subject to an acute stress such as coughing or jumping. This sudden movement in the urethra 3a may cause a temporary loss of continence as the passive and active mechanisms for preventing the flow of urine are overcome by the sudden movement and/or a change in fluid pressure.

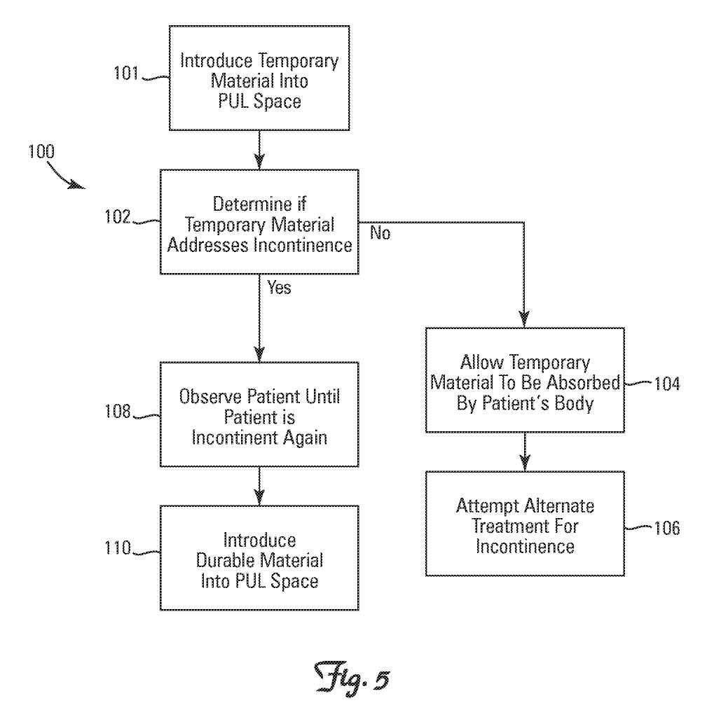

FIG. 4 shows a cross-sectional view of a female urinary tract in which a space filling material has been disposed in the pubourethral space 7a according to some embodiments of the present invention. As shown, deficient PULs 5a extend through the pubourethral space 7a. It is thought that the PULs 5a divide the pubourethral space 7a into distinct upper 8a and lower 9a spaces.

In some embodiments, the space filling material is disposed within both the upper 8a and lower 9a portions of the pubourethral space 7a around the PULs 5a. In some embodiments, the space filling material fills the entire, or substantially the entire, pubourethral space 7a and the space filling material surrounds a portion of, all of, or substantially all of, the urethra 3a. In yet other embodiments, the space filling material surrounds a portion of, all of, or substantially all of, the urethra 3a and also surrounds a portion of, all of, or substantially all of, the PULs 5a. In some embodiments, the space filling material restricts inferior/superior movement, medial/lateral movement and/or anterior/posterior movement of the urethra 3a within the pubourethral space 7a.

In other embodiments, the space filling material is disposed in one of the upper 8a or lower 9a portions of the pubourethral space 7a. In some such embodiments, the space filling material may exert sufficient pressure on the PULs 5a and/or the urethra 3a such that the PULs 5a and/or the urethra 3a become stretched in a downward direction (if the space filling material is placed in the upper portion 8a) or in an upward direction (if the space filling material is placed in the lower portion 9a). In some embodiments, this stretching causes the urethra 3a to experience increased support relative to the support provided by the unstretched, deficient PULs 5a.

In some embodiments, the space filling material that is disposed within the pubourethral space 7a is a viscous space filling material, while in other embodiments it is a structural space filling material.

In some embodiments in which the space filling material is a viscous space filling material, it is a gel or hydrogel. Examples include polyamines or polycarboxylate polymers with hydroxyphenyl compounds substituted on the polymers. Specific examples of such polymers are hyaluronic acid (HA), chondroitin, or collagen, or any other suitable polymers, for example any of those listed in U.S. Pat. No. 6,982,298, filed on Jan. 8, 2004; U.S. Pat. No. 7,368,502; and U.S. Patent Publication No. 2006/0084759, filed Jul. 7, 2005, all entitled “Hydroxyphenyl Cross-Linked Macromolecular Network and Applications Thereof,” all of which are herein incorporated by reference in their entirety.

An example of a suitable hydroxyphenyl compound is tyramine. In some embodiments, the gel or hydrogel is a modified or degradation resistant HA such as cross linked HA. In some embodiments, the HA is a tyramine-substituted HA that is prepared and cross-linked as described in U.S. Pat. Nos. 6,982,298 and 7,368,502 and in U.S. Publication No. 2006/0084759, which, as mentioned above, are herein incorporated by reference in their entirety. For example, the tyramine-substituted HA has a percent tyramine substitution of less than about 10%, less than about 5%, between about 4% and about 6%, between about 1.7% and about 4.7%, about 4.5% or about 5%. The percentage of tyramine-substituted HA in a pre-cross-linked solution (the solution prior to the tyramine-substituted HA being cross-linked) may be between about 6.25 mg/mL to about 100 mg/mL, from about 5 mg/mL to about 100 mg/mL, about 6.25 mg/mL, about 12.5 mg/mL, about 25 mg/mL, about 50 mg/mL, or about 100 mg/mL. The solvent for the pre-cross-linked solution may be any solution that is suitable for introduction into the human body, for example PBS, water, a tissue culture media, or a cell freezing solution, or any other suitable solvent, or combinations thereof. Additional suitable gels or hydrogels may also be prepared, as indicated in the above-mentioned patents and patent application. For example, other polymers such as polysaccharides, proteins, polylactic acid, and polyvinyl acetate may be used.

In some embodiments, the gel or hydrogel space filling material is a hydrophilic polymer that can form and maintain a shape. In some embodiments, the gel or hydrogel space filling material alters shape, for example when the patient moves or if the patient's anatomy changes. Exemplary shape-changing polymers are available from Landec Corporation of Menlo Park, Calif. Such polymers may have a T_{g} that is slightly below body temperature, which causes the polymers to be shape-changing when disposed within a body.

In other embodiments, the viscous space filling material is a viscous liquid or dense slurry. Examples include silicone oil, mineral oils, ionically cross linked HA (ferric HA), concentrated non-degradable polysaccharide, hydrogel particles, or modified starch particle. Some viscous liquids of dense slurries include a viscous liquid the may become more viscous upon reaching body temperature or upon absorbing water from surrounding tissues.

Other embodiments of the viscous space filling material include synthetic polymer particles that are delivered as a slurry, for example particles comprising a hydrogel, polypropylene, polyethylene, neoprene, polyvinyl acetate, polylactic acid, polyesters, polyurethanes, Landec polymers, ceramic material, metallic material and polytetrafluoroethylene. In yet other embodiments, the viscous space filling material is autologous or non-autologous fat.

In some embodiments, the viscous space filling material is injected directly into the pubourethral space (e.g., the entire pubourethral space, or one of the upper or lower pubourethral spaces, as mentioned above) through a needle or trocar, or is otherwise disposed in the pubourethral space via another laporascopic procedure or via an open surgical procedure. In some embodiments, the viscous space filling material is pre-formed, while in other embodiments the viscous space filling material is formed in situ by disposing precursor materials of the viscous space filling material into the pubourethral space and facilitating the formation of the viscous space filling material within the pubourethral space. Exemplary precursor materials and methods for forming suitable viscous space filling materials are provided in U.S. Pat. Nos. 6,982,298 and 7,368,502 and in U.S. Publication No. 2006/0084759, which, as mentioned above, are herein incorporated by reference in their entirety.

In addition, in some embodiments the space filling material is a structural space filling material such as a synthetic mesh, synthetic film, synthetic or natural particles, a self-expanding metallic structure, a synthetic polymer insert, a self-expanding mesh, tissues such as decellularized cadaveric tissues (such as facia, ligament or muscle), or other suitable support structure. In some embodiments, synthetic meshes comprise one or more of polyester, polypropylene, polyethylene, proteins, and polysaccharides. In some embodiments, the mesh, tape, film, particles or tissues are precompressed or are otherwise expandable. The mesh, tape, film, particles or tissues are introduced into the pubourethral space, for example via a needle or trocar with or without a carrier fluid or they are otherwise disposed in the pubourethral space via another laporascopic procedure or via an open surgical procedure. In some embodiments, the mesh, tape, film, particles or tissues are self-expanding, while in other embodiments the mesh tape, film, particles or tissues are expanded using expanders such as hydrophilic starches, unsaturated hydrogels, high osmolality polymers that expand when exposed to water, expanders that react to the pH typically present in the pubourethral space, or by using materials that react to heat (e.g., RF generated heat), or by using any other materials or techniques that facilitate the expansion of the structural space filling material.

In some embodiments in which the structural space filling material is a self-expanding metallic structure, the self-expanding metallic structure is a mesh or other geometric form that comprises a shape memory metal such as Nitinol. In some embodiments, the shape memory mesh or other geometric form is placed within the pubourethral space via a trocar, while in other embodiments the mesh or other geometric form is placed in the pubourethral space via another laporascopic procedure or via an open surgical procedure. In some embodiments in which the mesh or other geometric form comprises a one-way shape memory alloy such as Nitinol, the alloy has an austenite start temperature that is below body temperature, and in some embodiments both the austenite start and the austenite finish temperatures are below body temperature. When the mesh or other geometric form is placed in the body and heated above the austenite start temperature (or in some cases above the austenite start and finish temperatures), the mesh or other geometric form transforms from a first, contracted configuration to a second, expanded configuration. The second, expanded configuration is shaped and configured to restrict the mobility of the urethra.

The shape of the expanded structural space filling material may be any shape that is suitable for being placed in the pubourethral space and effectively filling the space in order to immobilize the urethra and/or the PULs. For example, the structural space filling material may have a round cross-section (e.g., it may be shaped like a cylinder or a sphere). In some embodiments, the structural space filling material expands between a wall of the pubourethral space and a PUL and/or the urethra. In some such embodiments, the structural space filling material places pressure on the PUL and/or the urethra, effectively removing slack from, and immobilizing, the PUL and/or urethra. A structural space filling material may be disposed in any portion of the pubourethral space, for example above the PULs and urethra, below the PULs and urethra, or both above and below.

In other embodiments in which the structural material is a solid synthetic insert, the insert comprises polypropylene, polyethylene, polyester, protein, polysaccharides, ceramics, or metal materials, or any other suitable materials. In some embodiments, the solid synthetic insert is inserted into the pubourethral space. For example, the solid synthetic insert is flexible or can be broken down into pieces and the solid synthetic insert is delivered through a trocar, or by using another laporascopic procedure. In other embodiments, the solid synthetic insert is placed in the pubourethral space using an open surgical technique.

In yet other embodiments, the structural space filling material is a container, sack, or balloon that is filled with a fluid or other inflation material. In some embodiments, the container, sack or balloon is inserted into the pubourethral space and inflated with an inflation material, for example a gas, a liquid, a gel, a polymerizable composition, or other suitable inflation material. In other embodiments, the container, sack, or balloon is filled with an expandable material, for example an expandable material that can be foamed (e.g., foamed via a chemical reaction, via a temperature change, and/or via introduction of a gas or other additional material). The container, sack or balloon may be prefilled with the expandable material, or the expandable material may be introduced after the container, sack, or balloon are disposed in a patient. In some embodiments, the balloon is inserted into the pubourethral space using a minimally invasive technique via a needle or trocar, while in other embodiments the balloon placed in the pubourethral space using another laporascopic technique or via an open surgical technique. The balloon remains in the pubourethral space in some embodiments, while in other embodiments the balloon is removed (e.g., after the inflation material has solidified or otherwise become sufficiently viscous to restrict mobility of the urethra).

In some embodiments, any of the structural space filling materials described above are used in conjunction with any of the viscous space filling materials described above. For example, in some embodiments, any of the structural space filling materials described above are disposed in the pubourethral space such that they at least partially restrict mobility of the urethra, and subsequently one or more of the viscous space filling materials is introduced into the pubourethral space. In other embodiments, the structural space filling material is introduced into the pubourethral space after the viscous space filling material has been disposed in the pubourethral space, or the structural space filling material and the viscous space filling material are disposed in the pubourethral space simultaneously. In some embodiments, the viscous space filling material surrounds all, substantially all, or a portion of, the structural space filling material in addition to surrounding all, substantially all, or a portion of, the urethra and/or the PULs.

As discussed above, a variety of methods may be used to dispose the space filling material in the pubourethral space. These include minimally invasive methods using needles or trocars for introduction of the space filling material into the pubourethral space. Other methods include open surgical methods in which an opening is formed in the abdomen of the patient and the space filling material is disposed in the pubourethral space through the opening. In some embodiments, the patient is provided a local and/or a general anesthetic for these procedures.

Also as discussed above, in some embodiments the space filling material has multiple components that are added to the pubourethral space. In some such embodiments, two or more fluid, gel, or hydrogel precursor components are added to the pubourethral space and together the precursor components form the space filling material. For example, a hydroxyphenyl substituted polyamine or polycarboxylate is placed in the pubourethral space together with a peroxidase enzyme and dilute hydrogen peroxide, and these compounds form diphenyl linkages, forming a cross-linked polymer matrix. Specifically, in one example a tyramine substituted collagen or HA polymer is introduced into the pubourethral space together with horseradish peroxidase and dilute hydrogen peroxide, which form a cross-linked collagen or HA polymer matrix. Exemplary combinations of chemical compounds, along with other compounds that may be used in the present invention, are described in U.S. Pat. Nos. 6,982,298 and 7,368,502, and U.S. Publication No. 2006/0084759, which, as mentioned above, are herein incorporated by reference in their entirety.

Further, in some embodiments in which precursor components are introduced to the pubourethral space in order to form the space filling material, the two or more precursor components are introduced simultaneously (either pre-mixed or blended together upon introduction to the pubourethral space), while in other embodiments they are introduced sequentially. When introducing the precursor materials separately, in some embodiments a single syringe is used and the two or more precursor materials are introduced sequentially through the single syringe. In other embodiments, more than one syringe is used to separately introduce the precursor materials, or a single syringe with multiple pathways is used.

The space filling materials are disposed in the pubourethral space in such a way to restrict mobility of the urethra, and in some cases restrict mobility of both the urethra and the PULs. As mentioned above, in some patients the restriction of mobility of the urethra, and in some cases the urethra and the PULs, facilitates urinary continence. In some embodiments, the space filling material surrounds the urethra, and in some cases both the urethra and the PULs.

Further, in some embodiments, the space filling material is disposed within the pubourethral space in such a way that the slack in the PULs is removed. In some such embodiments, the mobility of the urethra is restricted by the space filling material and also by the PULs that have had some or all of their slack removed. In addition, in some embodiments the space filling material may also effectively stretch, or take some slack out of the urethra itself, which in turn may facilitate continence. In some embodiments in which the urethra and/or the PULs are stretched and/or they have had slack removed, the stretching and/or slack removal is facilitated by making the pubourethral space larger, while in other embodiments the slack is removed by disposing the space filling material on one side of the PULs and/or urethra, as discussed above.

FIG. 5 shows a process diagram 100 for a method of addressing urinary incontinence according to some embodiments of the present invention. In this method, a temporary space filling material is introduced into the pubourethral space (block 101). For example, the temporary space filling material may be any of the polyamine or polycarboxylate polymers described above (e.g., HA, collagen, etc), with little or no cross-linking provided between polymer chains. Specifically, the temporary space filling material may be any of the polyamine or polycarboxylate polymers described above with or without hydroxyphenyl groups substituted on the polymers. The patient is then evaluated over a period of time (e.g., immediately in a doctor's office, over a period of several hours or less, for the remainder of the day, or for several days) in order to determine if the presence of the temporary material has addressed the urinary incontinence of the patient (block 102). The space filling material is temporary in that the patient's body will absorb the temporary material in a relatively short period of time (e.g., within several hours, within one day, within several days, within a week, or within a month). If the temporary space filling material does not sufficiently address the incontinence of the patient, the temporary space filling material is allowed to degrade within the patient's body (block 104), and an alternate treatment for the patient's incontinence is attempted (block 106). For example, bulking agents may be added to portions of the urethra, valves or other devices may be implanted in the urethra, urethral slings may be disposed around the urethra, or other alternate procedures may be performed on the patient.

If the temporary space filling material sufficiently addresses the incontinence of the patient, an optional step may be performed in which the patient is monitored for the return of the incontinent condition (block 108). The patient would be monitored for the return of incontinence for a period of time sufficient to allow the space filling material to be absorbed by he body. This step confirms that the space filling material is addressing the incontinence and also confirms that the temporary space filling material has been entirely or substantially degraded by the body. A permanent space filling material is then introduced into the pubourethral space (block 110), for example any of the viscous space filling materials, structural space filling materials, or combinations of viscous and structural space filling materials described above. As an alternative, the step indicated in block 108 may be omitted and the permanent space filling material introduced after a determination that the temporary space filling material has sufficiently addressed the urinary incontinence. The permanent space filling material is permanent in the sense that it does not degrade quickly within the patient. In some embodiments, the permanent space filling material does not significantly degrade within the lifetime of the patient, while in other embodiments the permanent space filling material does not significantly degrade over the period of one month, six months, one year, or two years.

In some embodiments, the polyamine or polycarboxylate polymers of the temporary space filling material has hydroxyphenyl substitution along the polymers but the polymers are introduced into the pubourethral space without horseradish peroxidase and/or hydrogen peroxide. As such, the hydroxyphenyl groups do not significantly cross-link with one another. The horseradish peroxidase and/or hydrogen peroxide may subsequently be added to cause the cross-linking of the polymer to cause the space filling material to become permanent, as discussed above.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

We claim:

1. A method for stabilizing a urethra in a female patient having stress urinary incontinence, the method comprising injecting precursor materials for a cross-linked hyaluronic acid (HA) into a pubourethral space and allowing the precursor materials to form a cross-linked hyaluronic acid such that the cross-linked hyaluronic acid fills substantially the entire pubourethral space.

2. The method of claim 1, wherein injecting the HA into the pubourethral space includes injecting two or more components into the space, the two or more components being precursor materials for the HA, and allowing the two or more components to cure.

3. The method of claim 1, wherein injecting the HA into the pubourethral space includes injecting a tyramine substituted HA, horseradish peroxidase and hydrogen peroxide into the pubourethral space.

4. The method of claim 1, wherein the HA forms a gel when cured.

5. The method of claim 1, wherein the HA fills the entire pubourethral space.

6. The method of claim 1, wherein the step of injecting the HA into the pubourethral space removes at least a portion of the slack in the PUL.

7. A method for stabilizing a urethra in a female patient having stress urinary incontinence wherein a pubourethral ligament (PUL) has been stretched, creating slack in the PUL, the method comprising injecting an amount of curable material into a pubourethral space such that the curable material restricts mobility of the urethra and curing the curable material, the curable material having a first consistency when injected and a second, thicker consistency after the curable material is allowed to cure.

8. The method of claim 7, wherein the curable material is a cross-linkable hyaluronic acid.

9. The method claim 7, wherein the curable material is a cross-linkable collagen.

10. The method of claim 7, wherein injecting the curable material into the pubourethral space includes injecting two or more components into the pubourethral space, the two or more components being precursor materials for the curable material, and allowing the two or more components to cure.

11. The method of claim 7, wherein injecting the curable material into the pubourethral space includes injecting a tyramine substituted hyaluronic acid, horseradish peroxidase and hydrogen peroxide into the pubourethral space.

12. The method of claim 7, wherein the curable material forms a gel when cured.

13. The method of claim 7, wherein the curable material fills the entire pubourethral space.

14. The method of claim 7, wherein the step of injecting the curable material into the pubourethral space removes at least a portion of the slack in the PUL.

15. A method for stabilizing a urethra in a female patient having stress urinary incontinence wherein a pubourethral ligament (PUL) has been stretched, creating slack in the PUL, the method comprising injecting a first hyaluronic acid (HA) into a pubourethral space, the first HA having a first level of cross-linking, monitoring the continence of the female patient and, if continence is improved with the introduction of the first HA, injecting a second HA into the pubourethral space after the first HA has at least partially degraded, the second HA having a second, greater level of cross-linking.

16. A method for stabilizing a urethra in a female patient having stress urinary incontinence, the method comprising disposing a space filling material in a pubourethral space in contact with the urethra and/or PUL such that the space filling material restricts mobility of the urethra.

17. The method of claim 16 wherein the space filling material is a structural space filling material.

18. The method of claim 17 wherein the space filling material is a self-expanding structure adapted for minimally invasive delivery to the pubourethral space.

19. The method of claim 16 wherein the space filling material is a viscous space filling material.

20. The method of claim 19 wherein the space filling material is a hydrogel.
